(19) United States
(12) Patent Application Publication
(10) Pub. No.: US 2021/0275645 A1
(43) Pub. Date: Sep. 9, 2021

(54) COMPOSITIONS AND METHODS FOR PREVENTING OR TREATING MACULAR DEGENERATION

(71) Applicant: GenoFocus, Inc., Yuseong-gu (KR)

(72) Inventors: Jae G. Pan, Sejong (KR); Eui J. Kim, Daejeon (KR); Jeong H. Kim, Seongnam-Si (KR); Do Y. Yum, Daejeon (KR)

(21) Appl. No.: 17/173,324

(22) Filed: Feb. 11, 2021

Related U.S. Application Data
(60) Provisional application No. 62/975,463, filed on Feb. 12, 2020.

(51) Int. Cl. A61K 38/44; A23L 29/00; A23L 33/135; C07K 16/22; A61K 38/17; A61P 27/02

(52) U.S. Cl. CPC A61K 38/446; A23L 29/06; A23L 33/135; C12Y 115/01001; A61K 38/179; A61P 27/02; C07K 16/22

(57) ABSTRACT

The present invention relates, in part, to methods of preventing or treating macular degeneration in a subject by administering a composition comprising a superoxide dismutase enzyme. The present invention also provides pharmaceutical and/or food compositions comprising a superoxide dismutase enzyme.

### CROSS-REFERENCE TO A RELATED APPLICATION

This application claims the benefit of priority to U.S. Provisional Application No. 62/975,463, filed on Feb. 12, 2020.

### SEQUENCE LISTING

The instant application contains a Sequence Listing which has been submitted in ASCII format via EFS-Web and is hereby incorporated by reference in its entirety. Said ASCII copy, created on May 10, 2021, is named GNX-00301_SL.txt and is 31,452 bytes in size.

### STATEMENT REGARDING PRIOR DISCLOSURE BY AN INVENTOR OR A JOINT INVENTOR UNDER 37 CFR 1.77(b)(6)

On Feb. 14, 2019, GenoFocus, the Assignee of the present invention, publicly disclosed a press release regarding a microbial-derived SOD enzyme for the treatment of age-related macular degeneration.

### TECHNICAL FIELD

The present invention provides methods of preventing or treating macular degeneration by administering an oral composition comprising superoxide dismutase (SOD). Also provided are pharmaceutical or food compositions comprising SOD for preventing or treating macular degeneration.

### BACKGROUND

Age-related macular degeneration (“AMD”) refers to the chronic, progressive degenerative pathology of the macula, which results in loss of central vision. Macular degeneration is a major cause of vision loss and irreversible central vision loss in adults over 50 years of age. More than 25 million people around the world suffer from AMD, and the number continues to grow rapidly due to increasing lifespans and the rapid growth of the elderly population. In addition, excessive use of electronic devices, such as smartphones and laptops, also contributes to the early onset and increased prevalence of macular degeneration in people today.

The most important causes of AMD are age-related atrophy and a decline in the function of retinal pigment epithelium (RPE), which plays a critical role in maintaining the homeostasis and physiological function of the retina that plays a key role in visual function. In addition, the age-related abnormal changes in Bruch's membrane and degeneration of choroidal capillaries are also thought to contribute to the etiology of AMD. Bruch's membrane functions as the basement membrane of the RPE, while choroidal capillaries are located on the outermost side of the neural retina and supply nutrients and oxygen to photoreceptor cells in which photoconversion occurs.

The age-related macular degeneration is largely classified into two categories: dry macular degeneration characterized by the degeneration and functional decline of RPE, Bruch's membrane, and choroidal capillaries; and wet macular degeneration which involves choroidal neovascularization (CNV) in addition to the symptoms of dry macular degeneration.

Wet macular degeneration occurs in 5 to 10% of patients with dry macular degeneration and can lead to acute blindness within months if left untreated. This is in contrast to dry macular degeneration in which vision deterioration progresses over a period of a few years, or even as long as about ten to twenty years.

In wet macular degeneration, there is a widespread decrease in oxygen partial pressure and nutrients across the subretinal space and the sub-retinal pigment epithelial (RPE) space, leading to ischemia in tissues accompanied by an inflammatory response.

In addition, the complement system, which plays an important role in oxidative stress and immune response, acts such that choroidal neovascularization (CNV) characteristically occurs in the subretinal space and the sub-retinal pigment epithelial (RPE) space, causing serous leakage and hemorrhage.

It is known that vascular endothelial cells, RPE cells, and inflammatory cells, such as monocytes and macrophages, are involved in the development of choroidal neovascularization.

Potential treatment for macular degeneration includes anti-angiogenic agents, such as a decorin peptide (PCT Publication No. WO 2005/116066; incorporated by reference) or a conjugate thereof (U.S. Patent Application No. 2009/0246133 A1; incorporated by reference). However, such agents have not shown to be effective against choroidal neovascularization or age-related macular degeneration.

The clinical standard of care for wet AMD is an antibody therapy against vascular endothelial growth factor (VEGF). While it has been effective in reducing blindness in many patients, the anti-VEGF antibody or a fragment thereof (e.g., aflibercept) has not been able to completely inhibit the formation and growth of choroidal neovascularization, in part due to its action being limited to the epithelial cells on the surface of neovascular vessels. Moreover, the antibody has not been effective in preventing the eventual loss of functional photoreceptor cells in the central foveal of the retina, resulting from disruption of the underlying RPE tissue. Furthermore, the anti-VEGF antibody is administered by intravitreal injection, causing fear and side effects in patients.

Accordingly, there is a great need for oral compositions and methods for effectively treating macular degeneration without the need for intravitreal injection.

### SUMMARY

The present invention is based, at least in part, on the discovery that oral administration of a superoxide dismutase (SOD) enzyme is effective in preventing and treating macular degeneration (e.g., wet macular degeneration).

SOD is an antioxidant enzyme that removes reactive oxygen species, a major cause of AMD. While attempts have been made in the past to administer orally the SOD enzyme to treat ocular diseases, it has not conferred a protective effect against light-induced oxidative stress (Sicard et al. (2006) Investigative Ophthalmology & Visual Science 47:2089). Similarly, the oral administration of GliSODin comprising mellon extracts enriched with SOD failed to protect against the onset of neovascular AMD in human (Hera et al. (2009) Investigative Ophthalmology & Visual Science 50:258). Moreover, GliSODin further comprises gliadin (a wheat protein), a known risk factor for celiac disease, thereby limiting the treatable patient population.

By contrast, the compositions and methods provided herein are surprisingly effective in preventing and treating wet macular degeneration. In some embodiments, by utilizing an isolated/purified SOD, the composition can be prepared using large quantities of SOD, without introducing excessive contaminating proteins. In some embodiments, by formulating with shellac, the SOD enzyme is protected from the gastric acid upon being administered orally. Thus, the compositions of the present disclosure can deliver orally an effective amount of active SOD, thereby eliminating a need for the intravitreal injection and simplifying the therapeutic modality of AMD treatment. In addition, in some embodiments, the SOD enzyme of the present disclosure is sourced from generally regarded as safe (GRAS) probiotics with proven safety. Thus, it eliminates any toxicity concerns. Importantly, the compositions and methods provided herein are highly effective in inhibiting CNV and restoring retinal function. Thus, these methods and oral compositions comprising SOD are highly effective in preventing or treating wet macular degeneration.

### BRIEF DESCRIPTION OF THE FIGURES

FIG. 1A-FIG. 1B show the extracted-ion chromatograms from an LC-MS analysis of purified GF-101. FIG. 1A shows Peptides T8 and T8 deamidated (T8(de)), and FIG. 1B shows Peptides T12 and T12 deamidated (T12(de).

FIG. 2A-FIG. 2B show the amino acid sequencing of Peptide T8(de) (upper panel) and Peptide 12(de) (lower panel) by LC MS/MS. FIG. 2A discloses SEQ ID NOS 30-33, respectively, in order of appearance. FIG. 2B discloses SEQ ID NOS 34-37, respectively, in order of appearance.

FIG. 3 shows the deamidation sites of GF-101 determined by peptide mapping and subsequent amino acid sequencing. FIG. 3 discloses SEQ ID NO: 38.

FIG. 4 shows the Base Peak Chromatogram from LC-MS analysis of GF-103.

FIG. 5 shows a schematic diagram of a mouse study to evaluate the in vivo effect of a pharmaceutical composition comprising SOD.

FIG. 6 depicts fundus fluorescein angiography images (upper panel), showing the changes in CNV lesions after administration of a test substance (10 U or 20 U of GF-101 (a composition comprising SOD); or 20 μg of aflibercept (AF; a positive control)). The bottom panel shows a graph showing CTF values.

FIG. 7 shows retinal tomography images obtained by an optical coherence tomography performed on laser-induced CNV mice administered with GF-101. The images show changes in the size of CNV lesions after the administration of GF-101.

FIG. 8 shows the size of CNV lesions calculated from retinal tomography images obtained by an optical coherence tomography, which was performed on laser-induced CNV mice administered with a test substance (10 U or 20 U of GF-101; or 20 μg of afilbercept (AF; a positive control)).

FIG. 9 shows the results of electroretinography on mouse CNV models that were irradiated with a laser and then subsequently treated with a test substance (10 U or 20 U of GF-101; 20 μg of afilbercept (AF; a positive control)); or phosphate buffered saline (PBS; a negative control).

FIG. 10 shows the changes in electroretinography b-wave amplitudes of mouse CNV models that were irradiated with a laser and then subsequently administered with a test substance (10 U or 20 U of GF-101; 20 μg of afilbercept (AF; a positive control)); or phosphate buffered saline (PBS; a negative control).

FIG. 11 shows the histological analysis of mouse CNV models that were irradiated with a laser and then administered with a test substance (10 U or 20 U of GF-101; 20 μg of afilbercept (AF; a positive control)); or phosphate buffered saline (PBS; a negative control). The tissues were stained with Haemotoxylin and Eosin (H & E) for observation.

FIG. 12 shows the results of a TUNEL assay demonstrating a decreased number of dead cells in retinas of the mouse CNV models irradiated with a laser and then treated with GF-101 (10 U or 20 U); Afilbercept (AF; a positive control); or phosphate buffered saline (PBS; a negative control).

FIG. 13 shows the ICAM-1, CD45, and F4/80 immunofluorescence (red) in laser induced CNV in mice.

FIG. 14 shows a schematic diagram of a rat study to evaluate the in vivo effect of a pharmaceutical composition comprising SOD.

FIG. 15 shows immunofluorescence staining using isolectin B4.

FIG. 16A and FIG. 16B show choroidal flat mounts of the laser-induced CNV. The CNV lesions were labeled with isolectin B4. The areas of CNV lesions were measured in each group. (FIG. 16A) Boxplot. Statistical analysis was performed by one way analysis of variances and followed by Dunnett's multiple comparison test as a post hoc test. **p<0.01 vs. G1. (FIG. 16B) A dose-dependent effect of the GF-103 treatment on reduction of the CNV area. Curve fitting was performed using GraphPad Prism (4PL).

FIG. 17A and FIG. 17B show quantification of HIF-1-alpha of retina after laser-induced choroidal neovascularization by ELISA. (FIG. 17A) Boxplot. Statistical analysis was performed by one way analysis of variances and followed by Dunnett's multiple comparison test as a post hoc test. **p<0.01 vs. G1. (FIG. 17B) A dose-dependent effect of the GF-103 treatment on the level of Hif-1-alpha. Curve fitting was performed using GraphPad Prism (4PL).

FIG. 18A and FIG. 18B show quantification of the VEGF level of retina after laser-induced choroidal neovascularization by ELISA. (FIG. 18A) Boxplot. Statistical analysis was performed by one way analysis of variances and followed by Dunnett's multiple comparison test as a post hoc test (*p<0.05; **p<0.01 vs. G1). (FIG. 18B) A dose-dependent effect of the GF-103 treatment on the level of VEGF. Curve fitting was performed using GraphPad Prism (4PL).

FIG. 19 shows the effects of GF-103 on fluorescein leakage in laser-induced choroidal neovascularization of rats. The “+” in the box represents the mean. Statistical analysis was performed by one way analysis of variances and was followed by pairwise t-test as a post hoc test. *p<0.05 vs. G1. **p<0.01 vs. G1.

### DETAILED DESCRIPTION

The present invention relates, in part, to compositions and methods for preventing and treating macular disorder (e.g., AMD, wet AMD). It is discovered herein that an oral composition comprising SOD is effective in inhibiting choroidal neovascularization (CNV) associated with wet AMD.

In certain aspects, provided herein are methods of treating or preventing macular degeneration by administering to a subject in need thereof an effective amount of a composition comprising a SOD enzyme. Also provided herein are methods of decreasing or inhibiting choroidal neovascularization (CNV) by contacting a retina with a composition comprising a SOD enzyme.

The methods may comprise the SOD enzyme that is isolated or purified. In some embodiments, the SOD enzyme is from a microorganism. In some such embodiments, the SOD enzyme is from a bacterium. In some embodiments, the SOD enzyme is from a bacterium generally regarded as safe (GRAS) for use as food. In some embodiments, the SOD enzyme is from a Bacillus species bacterium. In some such embodiments, the SOD enzyme is from the Bacillus amyloliquefaciens GF423 strain (KCTC 13222BP).

The methods may comprise various routes of administration. In some embodiments, the methods comprise administering orally the composition comprising the SOD enzyme. In some embodiments, the SOD enzyme is coated with shellac. In some embodiments, the methods comprise administering a pharmaceutical composition comprising the SOD enzyme.

The methods may result in a number of biological changes. For example, the methods may (i) decrease choroidal neovascularization (CNV); (ii) decrease cell death in the retina; (iii) decrease inflammation in the retina; (iv) decrease the expression of vascular endothelial growth factor (VEGF) in the retina; and/or (v) increase the retinal function.

The methods may treat various types and symptoms of macular degeneration. For example, the methods may treat an age-related macular degeneration (AMD). In addition, the methods may treat a wet AMD or a neovascular AMD.

The methods may comprise administering to a subject at least one additional agent that treats macular degeneration. In some embodiments, the at least one additional agent is ranibizumab or aflibercept.

The methods may treat various subjects. In some embodiments, the subject is a mammal, preferably a human, a dog, a cat, a mouse, or a rat. In preferred embodiments, the subject is a human.

In certain aspects, provided herein are pharmaceutical compositions comprising a SOD enzyme. Also provided herein are engineered polypeptides comprising a SOD enzyme. Further provided herein are medical or nutraceutical foods comprising a SOD enzyme.

The engineered polypeptides may comprise a mutation, e.g., deletion, insertion, or substitution of one or more amino acids that may or may not affect various aspects of the polypeptide, e.g., stability (in vitro, ex vivo, or in vivo), homogeneity, and/or structural conformation changes. In some embodiments, the engineered polypeptides may comprise a truncation. In some embodiments, the engineered polypeptides may comprise a heterologous nucleic acid (e.g., HIS tag, HA tag, myc tag, other tags that are well known in the art, GFP, and/or Fc domain of an antibody) that may be useful, e.g., in purification, detection (in vitro, ex vivo, or in vivo), or extension of in vivo stability of said polypeptides.

The compositions or food may comprise the SOD enzyme that is isolated or purified. In some embodiments, the SOD enzyme is from a microorganism. In some such embodiments, the SOD enzyme is from a bacterium. In some embodiments, the SOD enzyme is from a bacterium generally regarded as safe (GRAS) for use as food. In some embodiments, the SOD enzyme is from a Bacillus species bacterium. In some such embodiments, the SOD enzyme is from the Bacillus amyloliquefaciens GF423 strain (KCTC 13222BP).

The compositions or food may comprise various forms. In some embodiments, the composition is an oral composition. In some embodiments, the food is ingested orally. In some embodiments, the SOD enzyme is coated with shellac.

The compositions or food may result in a number of biological changes. For example, the compositions or food may (i) decrease choroidal neovascularization (CNV); (ii) decrease cell death in the retina; (iii) decrease inflammation in the retina; (iv) decrease the expression of vascular endothelial growth factor (VEGF) in the retina; (v) decreased the expression of Hypoxia-inducible factor 1-alpha (HIF-1-alpha) in the retina; and/or (vi) increase the retinal function.

The compositions or food may comprise at least one additional agent that treats macular degeneration or inhibits CNV. In some embodiments, the at least one additional agent is ranibizumab or aflibercept.

In some embodiments, the SOD enzyme of the engineered polypeptides, compositions (e.g., pharmaceutical composition, oral composition), or medical or nutraceutical foods of the present disclosure, or the SOD enzyme used in the methods described herein, may comprise the amino acid sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to the sequence set forth in SEQ ID NO: 1.

In some embodiments, the SOD enzyme of the engineered polypeptides, compositions (e.g., pharmaceutical composition, oral composition), or medical or nutraceutical foods of the present disclosure, or the SOD enzyme used in the methods described herein, may comprise the amino acid sequence set forth in SEQ ID NO: 1, wherein the amino acid residue Asn74 and/or Asn137 is deleted or substituted.

In some embodiments, the SOD enzyme of the engineered polypeptides, compositions (e.g., pharmaceutical composition, oral composition), or medical or nutraceutical foods of the present disclosure, or the SOD enzyme used in the methods described herein, may comprise the amino acid sequence set forth in SEQ ID NO: 1, wherein the amino acid residue Asn74 and/or Asn137 is substituted with Asp74 and/or Asp137.

In some embodiments, the SOD enzyme of the engineered polypeptides, compositions (e.g., pharmaceutical composition, oral composition), or medical or nutraceutical foods of the present disclosure, or the SOD enzyme used in the methods described herein, may comprise the amino acid sequence set forth in SEQ ID NO: 1.

In some embodiments, the engineered polypeptides, compositions (e.g., pharmaceutical composition, oral composition), or medical or nutraceutical foods of the present disclosure may be administered orally, intravenously, intraocularly, or intramuscularly.

In preferred embodiments, the engineered polypeptides, compositions (e.g., pharmaceutical composition, oral composition), or medical or nutraceutical foods of the present disclosure may be administered orally.

In certain aspects, provided herein are kits comprising the engineered polypeptides, the pharmaceutical compositions, or the medical or nutraceutical foods of the present disclosure.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow therapy to perform its intended function. Examples of routes of administration include oral administration, sublingual administration, and intravitreal administration. As used herein, the term “age-related macular degeneration” or “AMD” includes early, intermediate, and advanced AMD, and also includes both dry macular degeneration, geographic atrophy, and wet macular degeneration, also known as neovascular or exudative AMD.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

As used herein, the terms “prevent,” “preventing,” and “prevention” are art-recognized, and when used in relation to a medical condition such as a loss of vision, or a disease such as macular degeneration, is well understood in the art, and include administration of a composition which reduces the frequency of, or delays the onset of, symptoms of a medical condition (e.g., blurry vision or a loss of vision) in a subject relative to a subject which does not receive the composition.

The term “subject” or “patient” refers to any healthy or diseased animal, mammal or human, or any animal, mammal or human. In some embodiments, the subject is afflicted with macular degeneration (e.g., neovascular macular degeneration). In various embodiments of the methods of the present invention, the subject has not undergone treatment. In other embodiments, the subject has undergone treatment.

As used herein, the term “therapeutically effective amount” of the composition or agent refers to an amount of an agent which provides the desired effect, such as reducing, preventing or slowing the progression of physical changes associated with macular degeneration in the eye, or reducing, preventing or slowing the progression of symptoms (e.g., accumulation of drusen, abnormal blood vessel growth in the eye, abnormal fluid in the eye, blood and protein leakage, etc.) resulting from them. The exact amount of agent required may vary from subject to subject depending on the species, age and general condition of the subject, mode of administration, and the like. However, an appropriate “effective amount” in any individual case may be determined by one of ordinary skill in the art using routine experimentation.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

### Macular Degeneration

The pathogenesis of AMD is still incompletely understood due to various factors. Aging of retinal pigment epithelial layer (RPE) cells and Bruch's membrane, impaired blood flow in the vascular membrane of the eye, retinal exposure to ultraviolet light and blue light, and genetic predisposition are believed to play an important role in the development of AMD.

The loss of RPE cells, which appears in the early stage of AMD, is mainly due to oxidative stress, which results from weakening of the antioxidant cell defense system or increased concentration of reactive oxygen species, and thus effective removal of reactive oxygen species may be essential for prevention and treatment of AMD.

From 1% to 5% of the total oxygen consumption in the body is converted into reactive oxygen species (ROS), which are the major source of oxidative stress. An imbalance between routine production and detoxification of reactive oxygen species (“ROS”) such as peroxides and free radicals can result in oxidative damage to cellular structures and machinery. The human retina consumes a large amount of oxygen, and in particular, retinal pigment epithelial cells produce a large amount of reactive oxygen species because these cells phagocytose the visual cell outer segment. In addition, intracellular reactive oxygen species are also produced through the mitochondrial electron transport system. Oxidative stress-induced retinal pigment epithelial cells undergo induced apoptosis or show changes such as mitochondrial DNA damage, increased vascular endothelial growth factor (VEGF), decreased antioxidant enzymes, and increased inflammatory responses.

### Superoxide Dismutase (SOD)

Superoxide dismutase (SOD) is an enzyme that alternately catalyzes the dismutation of the superoxide (O₂—) radical into either ordinary molecular oxygen (O₂) or hydrogen peroxide (H₂O₂). Thus, SODs play a key role in decreasing oxidative stress by removing reactive oxygen species. SODs are widely distributed in prokaryotic and eukaryotic cells and have been classified into four families based on their different types of metal centers [copper/zinc, nickel, manganese, and iron]. Manganese-containing SODs [Mn-SODs] are widely present in many bacteria, chloroplasts, mitochondria, and cytosol of eukaryotic cells. The SOD enzyme derived from B. amyloliquefaciens GF423 strain (KCTC 13222BP) is a Mn-SOD and has the amino acid sequence of SEQ ID NO: 1.

### Isolation/Purification of SOD

An “isolated” or “purified” SOD or biologically active portion thereof is substantially free of cellular material or other contaminating proteins from the cell or tissue source from which the enzyme is derived. The language “substantially free of cellular material” includes preparations of a polypeptide, in which the protein is separated from cellular components of the cells from which it is isolated or recombinantly produced. In some embodiments, the language “substantially free of cellular material” includes preparations of protein, having less than about 30% (by dry weight) of non-desired protein, more preferably less than about 20% of non-desired protein, still more preferably less than about 10% of non-desired protein, and most preferably less than about 5% non-desired protein.

SOD can be isolated or purified from various sources, including natural or recombinant hosts. For example, SOD having an activity of preventing or treating macular degeneration disease can be extracted from the culture supernatant of the B. amyloliquefaciens GF423 strain. First, a culture can be obtained by culturing the B. amyloliquefaciens GF423 strain in various types of media. In some embodiments, a complex medium (pH 6.0 to 7.0) is used to grow the bacteria at 25 to 42° C. for 1 to 4 days. Other suitable media for culturing the B. amyloliquefaciens GF423 strain include LB (Luria-Bertani) medium, ISP (International Streptomyces Project) medium, NA (nutrient agar) medium, BHI (brain heart infusion agar) medium, SDA (sabouraud dextrose agar) medium, PDA (potato dextrose agar) medium, NB (nutrient broth) medium, and the like. In preferred embodiments, LB medium, ISP medium, BHI medium, SDA medium, or NB medium may be used.

SOD may also be sourced from other natural or recombinant hosts using the information provided in databases such as PubMed or BRENDA (world wide web at brenda-enzymes.org).

The SOD is preferably purified by the following purification method but is not limited thereto. A culture obtained by culturing the B. amyloliquefaciens GF423 strain is centrifuged to collect the culture supernatant. The supernatant fraction is pretreated by solid-phase extraction and then isolated and purified by chromatography. Various modes of chromatography may be used to purify SOD. In preferred embodiments, a hydrophobic interaction chromatography is used.

### Pharmaceutical Compositions

The composition of the present invention may further comprise a conventional pharmaceutically acceptable carrier or excipient. In addition, the SOD enzyme derived from the B. amyloliquefaciens GF423 strain may be formulated with various additives, such as a binder, a coating agent and the like, which are pharmaceutically commonly used.

The pharmaceutical composition containing the SOD according to the present invention may contain a pharmaceutically acceptable carrier. For oral administration, the pharmaceutically acceptable carrier may include a binder, a lubricant, a disintegrant, an excipient, a solubilizer, a dispersing agent, a stabilizer, a suspending agent, a coloring agent, a flavoring agent, and the like. For topical administration, the pharmaceutically acceptable carrier may include a base, an excipient, a lubricant, a preservative, and the like. The pharmaceutical composition of the present invention may be formulated into a variety of dosage forms in combination with the aforementioned pharmaceutically acceptable carriers. For example, for oral administration, the pharmaceutical composition may be formulated in solid or liquid dosage forms such as tablets, troches, capsules, elixirs, suspensions, syrups, wafers, or the like. In addition, the pharmaceutical composition may be formulated into solutions, suspensions, tablets, capsules, sustained-release preparations, or the like.

Meanwhile, examples of the carrier, excipient, and diluent suitable for formulation may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, mineral oil, or the like. In addition, the pharmaceutical composition may further contain a filler, an anti-agglutinating agent, a lubricating agent, a wetting agent, a flavoring agent, an emulsifying agent, a preservative, or the like.

In the method of the present invention, the SOD enzyme may be coated with shellac. When the SOD is administered orally, a problem may arise in that the activity of the SOD is reduced rapidly in the gastrointestinal tract, leading to a decrease in the bioavailability and efficiency thereof. This problem is further exacerbated by the difficulty of delivering the SOD to the particular cell location where the SOD is most effective. Thus, in the method of the present invention, the SOD enzyme may be coated in a solution. Specifically, a purified solution and a shellac-containing solution are mixed with each other, and then freeze-dried. This freeze-dried sample may be powdered and stored at about 4° C. until use. Examples of coatings suitable for use in the present invention include shellac, ethyl cellulose, hydroxypropyl methylcellulose, hydroxypropyl methylcellulose phthalate, zein, Eudragit, and combinations thereof.

### Dose

The dose of the pharmaceutical composition of the present invention, which contains the SOD produced from the B. amyloliquefaciens GF423 strain, may be suitably determined in consideration of the purpose of treatment or prevention, the type of patient to be prevented or treated, the patient's condition, weight, age or sex, etc. For example, the composition of the present invention may contain, as an active ingredient, the SOD produced by the B. amyloliquefaciens GF423 strain in a therapeutically effective amount or at a nutritionally effective concentration. Preferably, the composition may contain the SOD in an amount of 2 to 300 U/mg, based on the total weight of the composition.

### A Medical or Nutraceutical Food

Still another aspect of the present invention provides a food, particularly a nutraceutical food, or medical food, for preventing or ameliorating macular degeneration and a degenerative decline in eye function, the food containing a SOD derived from the B. amyloliquefaciens GF423 strain. The SOD has the amino acid sequence of SEQ ID NO: 1.

As used herein, the term “nutraceutical food” or “medical food” means a food prepared with such a raw material or a component that is likely to be beneficial for function of the human body, which is defined by Ministry of Food and Drug Safety as the food to maintain or improve health by maintaining the normal function or by activating the physiological function of the human body, but not always limited thereto and does not exclude any conventional health food in its meaning.

The nutraceutical or medical food of the present invention may be prepared and processed in the form of tablets, capsules, powders, granules, liquids, pills, or the like, for the purpose of preventing or ameliorating macular degeneration. Conventional additives include, for example, chemical synthetic additives, such as ketones, glycine, calcium citrate, nicotinic acid, cinnamic acid, and the like; natural additives, such as persimmon color, licorice extract, crystalline cellulose, kaoline pigment, guar gum, and the like; and mixed formulations, such as L-sodium glutamate formulations, alkali additives for noodles, preservative formulations, tar color formulations, and the like. For example, a nutraceutical food in the form of a tablet may be prepared by granulating a mixture of the active ingredient SOD of the present invention with an excipient, a binder, a disintegrating agent and other additives by a conventional method, and then adding a lubricant, or the like thereto, followed by compression molding, or directly compression-molding the mixture. In addition, the nutraceutical food in the form of a tablet may contain a corrigent, or the like, if necessary.

Among nutraceutical foods in the form of a capsule, a hard capsule formulation may be prepared by filling a hard capsule with a mixture of the active ingredient SOD or bacterial strain powder of the present invention with an additive, such as an excipient. A soft capsule formulation may be prepared by filling a mixture of the SOD or the strain powder with an additive, such as an excipient, into a capsule such as a gelatin capsule. The soft capsule formulation may, if necessary, contain a plasticizer, such as glycerin or sorbitol, a coloring agent, a preservative, or the like.

A nutraceutical food in the form of a pill may be prepared by molding a mixture of the active ingredient SOD of the present invention with an excipient, a binder, a disintegrant, and the like by a known method. The pill formulation may, if necessary, be coated with white sugar or other coating agent or may also be surface-coated with a substance such as starch or talc.

### Conjoint or Combination Therapy

The methods and compositions provided herein may contain a single such molecule or agent (e.g., SOD) or any combination of the agents that are useful in treating macular degeneration. A single active agent described herein can be combined with one or more other pharmacologically active compounds known in the art according to the methods and compositions provided herein. It is believed that certain combinations work synergistically in the treatment of macular degeneration (e.g., wet AMD) or in the inhibition of CNV. Second active agents can be large molecules (e.g., proteins) or small molecules (e.g., synthetic inorganic, organometallic, or organic molecules).

In some embodiments, at least one additional therapy that may be combined with SOD is an agent that can treat macular degeneration. In some embodiments, the agent is approved by the U.S. Food and Drug Administration. In some such embodiments, the agent is afilbercept, an inhibitor of VEGF. In other such embodiments, the agent is ranibizumab, another inhibitor of VEGF. In some embodiments, the compositions provided herein are used as a primary treatment. In other embodiments, the compositions are used as adjuvant therapy. In some such embodiments, the compositions may be administered to a subject before, concurrently, or after the administration of the primary treatment.

### Kits

The present invention also encompasses kits. For example, the kit can comprise an engineered polypeptide of the present disclosure, a pharmaceutical composition as described herein, medical or nutraceutical food as described herein, a combination therapy including e.g., at least one additional agent that treats macular degeneration or decreases or inhibits CNV, for example, ranibizumab or aflibercept, or any combination thereof, packaged in a suitable container and can further comprise instructions for using such reagents. The kit may also contain other components, such as administration tools packaged in a separate container.

### EXAMPLES

### Example 1. Strain Isolation and Identification

1) 16S rRNA Analysis

From Bacillus polyfermenticus purchased from Bi-Nex Co., Ltd., a strain was isolated (“the Strain”), and the Strain was identified and characterized as described below.

To characterize the Strain, a morphological and biochemical examination was performed. The morphological examination of the Gram stained bacteria indicated that the Strain was a Gram-positive bacillus. In addition, observation under a phase contrast microscope showed that the Strain formed endospores.

To determine the identity of the Strain, 16s rRNA sequencing was performed as follows. The genome of the Strain was purified (Sambrook, J. et al.: “Molecular Cloning. A Laboratory Manual, 3rd ed.,” 2001, Cold Spring Harbor Press), and sequenced using Illumina HiSeq PE100. Nine copies of the 16S rRNA gene (SEQ ID NOs: 2 to 10) were found. Among the 16S rRNA genes, BPJGP_r00130 (SEQ ID NO: 7) and BPJGP_r00160 (SEQ ID NO: 8) showed the same nucleotide sequence, but other 16S rRNA genes showed different nucleotide sequences. Thus, the Strain had eight 16S rRNA genes with distinct nucleotide sequences.

With 9 copies of the 16S rRNA gene, analysis for the genus identification was performed using the following database and softwares: The Ribosomal Database Project's Classifier (Wang, Q. et al., Appl Environ Microbiol., 73:5261-5267 (2007)), Living Tree Project's Aligner (Pruesse, E. et al., Bioinformatics, 28:1823-1829 (2012)), and EzTaxon database's Identity (Kim, O. S. et al., Int J Syst Evol Microbiol., 62:716721 (2012)). The Strain was identified to be a member of the genus Bacillus according to all the softwares listed above, with a confidence interval of 95% or more.

Species level identification of the isolated strain was performed using the EzTaxon database's Identity (Kim, O. S. et al., Int J Syst Evol Microbiol., 62:716721 (2012)). Although there is currently no international standard for the identity threshold of 16S rRNA for species level identification, 99% is the highest value of the most widely accepted thresholds (Yarza, P. et al., Nature Rev. Microbiol., 12: 635645 (2014)). Accordingly, the 99% threshold was used as a search standard. In addition, since the Strain had eight distinct 16S rRNA genes, a search was performed for each of the 16S rRNA genes. Among the found reference strains, the commonly found reference strains were selected. The search identified 80 different reference strains belonging to different species. This result is consistent with previous studies indicating that species belonging to the genus Bacillus cannot be distinguished using only the homology of 16S rRNA genes (Janda J. M. & Abbott S. L., J Clin Microbiol., 45:2761-2764 (2007); Maughan H. & Van der Auwera G., Infect Genet Evol., 11:789-797 (2011)).

Thus, in order to determine the identity of the Strain, a genome-based classification was performed. The homology between the Strain and the 80 strains identified above was analyzed using the in silico DNA-DNA Hybridization (DDH; Auch A. F. et al., Stand Genomic Sci., 28:117-234(2010)), and the reference strains showing a homology of greater than 70% were selected. Two reference strains were found in the analysis (see Table 1 below), and their ANI (the average nucleotide identity) and AAI (the average amino acid identity) at the genomic level with respect to the Strain were verified (Rodriguez-R L. M. & Konstantinidis K. T., https://doi.org/10.7287/peerj.preprints.1900v1 (2016)).

Table 1 below shows the analysis of the 16S rRNA gene, DDH, ANI and AAI of three strains, which showed the highest homology with the Strain in the DDH analysis.

TABLE 1

| | Bacillus | Bacillus | Bacillus | Criteria for |
|---|---|---|---|---|
| | amyloliquefaciens | amyloliquefaciens | subtilis | species |
| | plantarum | amyloliquefaciens | spizizenii | classification |
| Reference | FZB42 | DSM7 | NRRLB-23049 | |
| strain | | | | |
| 16S | 99.67 to 99.73% | 99.46 to 99.66% | 99.18 to 99.39% | 98.65% |
| rRNA | | | | |
| DDH | 92.10% | 78.60% | 32.70% | 70% |
| ANI | 98.65% | 93.59% | 80.04% | 95% |
| AAI | 98.79% | 95.09% | 79.88% | 95% |

Comparison of the 16S rRNA genes described above identified the Strain as a microorganism belonging to B. amyloliquefaciens. The Strain was named Bacillus amyloliquefaciens GF423 and deposited with the Korean Collection for Type Cultures (KCTC), a patent strain depository authority, on Mar. 6, 2017, under accession number KCTC 13222BP.

### Example 2. Isolation/Purification of Superoxide Dismutase (SOD) from Bacillus amyloliquefaciens GF423

2.1 Culturing of Bacillus amyloliquefaciens GF423 Strain

For culturing of the Bacillus amyloliquefaciens GF423 strain, a single colony formed in LB agar medium (Luria-Bertani (LB) agar; 10 g/L tryptophan, 5 g/L yeast extract, 10 g/L NaCl, 15 g/L agar) was inoculated into 30 mL of LB medium and cultured at 37° C. for 12 hours. The seed culture was inoculated again into 3 L of LB medium containing 1 mM manganese sulfate (MnSO₄) and was cultured at 37° C. for 20 hours. Then, a portion of the culture was used for the separation of SOD. The remaining portion was diluted at 10¹¹ CFU/mL in phosphate buffered saline (PBS, 10 mM sodium phosphate, 130 mM sodium chloride, pH 7.4) and sonicated, and then the supernatant was collected by centrifugation, filtered through a filter having a pore size of 0.45 μm, freeze-dried, and then stored at −20° C. until use in an in vivo experiment.

2.2 Isolation and Purification of Superoxide Dismutase

The culture of the B. amyloliquefaciens GF423 strain was centrifuged at 3,578×g at 4° C. for 20 minutes and the supernatant was collected and concentrated 10-fold by ultrafiltration (MWCO 10,000). Ammonium sulfate was added to 300 mL of the concentrated supernatant to a saturation degree of 60% with stirring at 4° C., followed by stirring for 30 minutes. Then, the supernatant was collected by centrifugation at 3,578×g for 30 minutes, and loaded onto a HiPrep™ Phenyl HP 16/10 column equilibrated with 50 mM potassium phosphate (pH 7.5) containing 2 M ammonium sulfate. Next, elution was performed using 50 mM potassium phosphate (pH 7.5) containing 2 M to 0.1 M ammonium sulfate. The SOD-containing fraction was collected, concentrated by UF (MWCO 10,000), and desalted by dialysis with 50 mM potassium phosphate (pH 7.5). The activity of the SOD was analyzed using a SOD assay kit (Cayman Chemical, Michigan, USA). One unit of SOD activity is defined as the amount of enzyme that inhibits superoxide radicals by 50%. The activity of the purified SOD enzyme was 2231.12±269 U/mg, and the molecular weight of the SDS was about 22,000 Dalton. The SOD derived from the B. amyloliquefaciens GF423 strain was designated as GF-101.

The SOD derived from the B. amyloliquefaciens GF423 (hereinafter GF-101) was coated with the natural coating agent shellac. Shellac was dissolved in 50 mM potassium phosphate (pH 7.0) buffer, mixed with a purified solution of the SOD, and freeze-dried. The freeze-dried sample was in a powder form and stored at 4° C.

### Example 3. A Variant SOD, GF-103

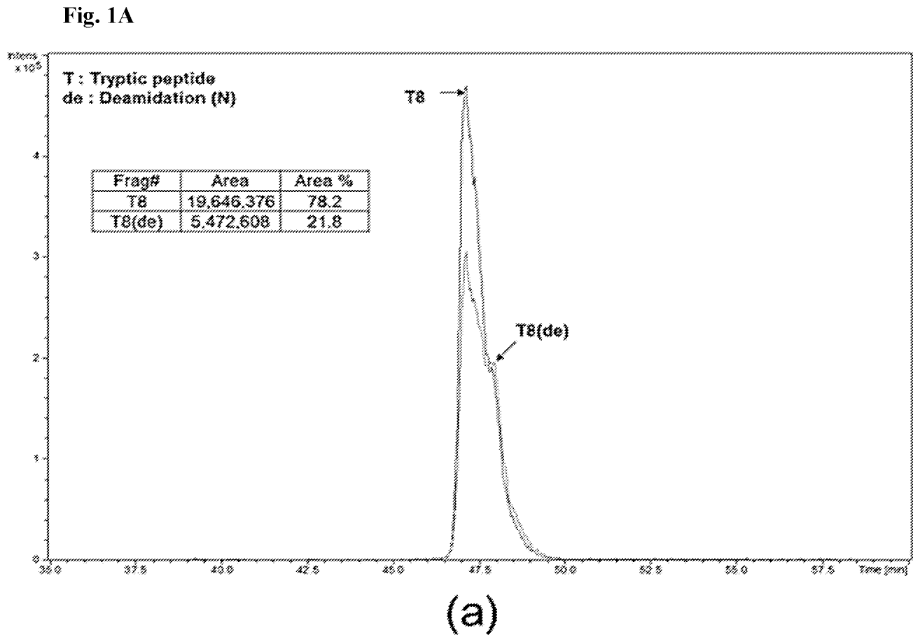

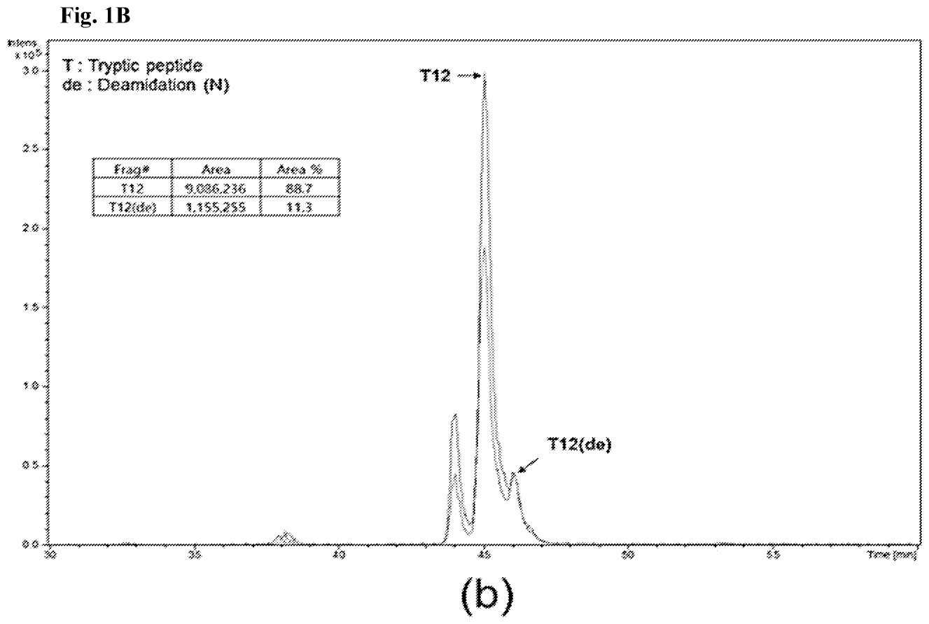

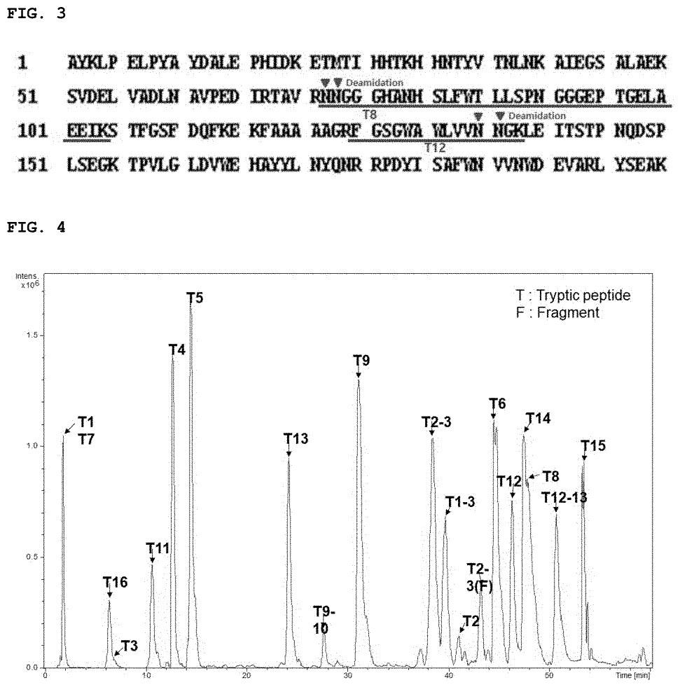

Deamidation of some populations of Asn74 and Asn137 residues in the purified GF-101 was found by peptide mapping with trypsin digest (FIG. 1) and amino acid sequencing analysis (FIG. 2): 21.8% for Asn74 and 11.3% for Asn137. FIG. 3 summarizes the deamidation sites and the peptides harboring the sites with the amino acid sequence of GF-101. The two Asn residues were substituted for Asp to improve the homogeneity of the purified enzyme. The variant SOD was designated as GF-103. Peptide mapping of GF-103 (FIG. 4) showed that there was no unexpected peptide. Subsequent amino acid sequencing of the peptides (Table 2) confirmed the results of peptide mapping. The substitutions of Asn to Asp did not affect enzyme activity and/or stability.

TABLE 2

| Theoretical Mass (T) | | | | | GF103_190 [symbol] 11 | | |
|---|---|---|---|---|---|---|---|
| Frag^{#} | Res^{#} | Modification | m/z | Charge | m/z | RT | Sequencing |
| T1 | 1-3 | | 381.21 | 1 | 381.21 | 1.8 | AYK |
| T2 | 4-20 | | 662.01 | 3 | 662.00 | 41.0 | LPELPYAYDALEPHIDK |
| T3 | 21-29 | | 549.27 | 2 | 549.27 | 6.8 | ETMTIHHTK |
| T2-3 | 4-29 | Miss-cleavage | 613.31 | 5 | 613.31 | 3 [symbol] .3 | LPELPYAYDALEPHIDKETMTIHHTK |
| T2-3(F) | 4-23 | Fragment, Miss-cleavage | 782.38 | 3 | 782.38 | 4 [symbol] .1 | LPELPYAYDALEPHIDKETM |
| T1-3 | 1-29 | | 856.94 | 4 | 856.98 | 38.6 | AYKLPELPYAYDALEPHIDKETMTIHHTK |
| T4 | 30-40 | | 447.56 | 3 | 447.56 | 12.7 | HHNTYVTNLNK |
| T5 | 41-50 | | 494.77 | 2 | 494.77 | 14.5 | AIEGSALAEK |
| T6 | 51-67 | | 927.98 | 2 | 927.97 | 44.4 | SVDELVADLNAVPEDIR |
| T7 | 68-71 | | 446.27 | 1 | 446.27 | 1.9 | TAVR |
| T8 | 72-104 | | 851.91 | 4 | 851.90 | 47.7 | NDGGHANHSLFWTLLSPNGGGEPTGELAEEIK |
| T9 | 105-114 | | 582.27 | 7 | 582.27 | 31.0 | STFGSFDQFK |
| T10 | 115-116 | | 276.16 | 1 | N.D | N.D | EK |
| T9-10 | 105-116 | Miss-cleavage | 474.23 | 3 | 474.23 | 27.6 | STFGSFDQFKFK |
| T11 | 117-124 | | 367.70 | 2 | 367.70 | 10.7 | FAAAAAGR |
| T12 | 125-138 | | 768.39 | 2 | 767.38 | 46.2 | FGSGWAWLVVNDGK |
| T13 | 139-155 | | 908.45 | 2 | 908.45 | 24.2 | LEITSTPNQDSPLSEGK |
| T12-13 | 125-155 | Miss-cleavage | 1111.55 | 3 | 1111.54 | 50.6 | FGSGWAWLVVNDGKLEITSTPNQDSPLSEGK |
| T14 | 156-175 | | 817.74 | 3 | 817.74 | 47.3 | TPVLGLDVWEHAYYLNYQNR |
| T15 | 176-194 | | 779.72 | 3 | 779.71 | 5 [symbol] .3 | RPDVISAFWNVVNWDEVAR |
| T16 | 195-200 | | 355.69 | 2 | 355.69 | 6.4 | LYSEAK |

[symbol]

indicates data missing or illegible when filed

Table 2 discloses SEQ ID Nos 11-29, respectively, in order of appearance.

### Example 4. Evaluation of Choroidal Neovascular Inhibitory Effect of Superoxide Dismutase (SOD) Derived from Bacillus amyloliquefaciens GF423

4.1. Experimental Animals and Construction of Choroidal Neovascular (CNV) Models

Animal experiments were performed in accordance with the Animal Use and Care Protocol of the Institutional Animal Care and Use Committee (IACUC). C57BL/6 mice were purchased from Koatech Co., Ltd., and acclimated for 14 days. Then, the mice were raised for 17 days at an average temperature of 19° C. to 25° C., a humidity of 40% to 60% and an average illuminance of 150 to 300 lux with a 12-hr light/12-hr dark cycle. The mice were given feed and water ad libitum daily.

7-week-old C57BL/6 mice were anesthetized with a mixture of ketamine hydrochloride (40 mg/kg) and xylazine hydrochloride (10 mg/kg), and then the Bruch's membrane of the mouse eye was irradiated with a diode green laser (532 nm, 150 mW, 0.1 sec, 50 μM), thereby inducing choroidal neovascularization.

4.2. Administration of Test Substances

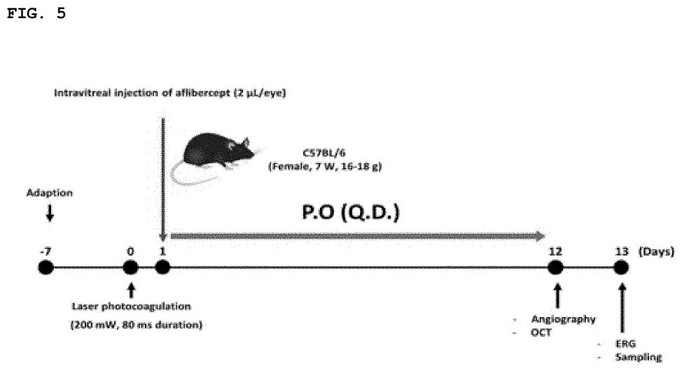

Experimental animals, grouped as described below, were irradiated with a laser. One day after laser photocoagulation, 100 μl of each of GF-101 (10 U) and GF-101 (20 U) dissolved in PBS, was administered thereto orally daily for 12 days. A positive control aflibercept (AF) (20 μg) was administered once by intravitreal injection on day 1 (FIG. 5). Aflibercept is a product approved by the US Food and Drug Administration (FDA) for use as an agent for treating age-related macular degeneration. To a negative control group and a CNV-induced group (test group II), PBS as a placebo was administered in the same manner as the GF-101 group. GF-101 is SOD derived from the B. a. GF423 strain.
Test group I (NC): normal control group.
Test group II: group administered with PBS after CNV induction.
Test group III (PC): group administered with aflibercept.
Test group V: group administered with GF-101 (10 U) after CNV induction.
Test group VI: group administered with GF-101 (20 U) after CNV induction.
GF-101 (20 U): Aliquots of GF-101 were stored in a test substance freezer (4° C.) of the test institute, and then taken out once a day immediately before administration. A solution having a concentration of 200 U/mL was prepared by mixing 26.6 mg of GF-101 with 2 mL of PBS, and 100 μl of the solution was administered orally to each animal.
GF-101 (10 U): A solution having a concentration of 200 U/mL was prepared by diluting GF-101 (200 U/mL) two-fold, and 100 μL of the solution was administered orally to each animal.

4.3. Animal Tests

Fundus Fluorescein Angiography (FFA)

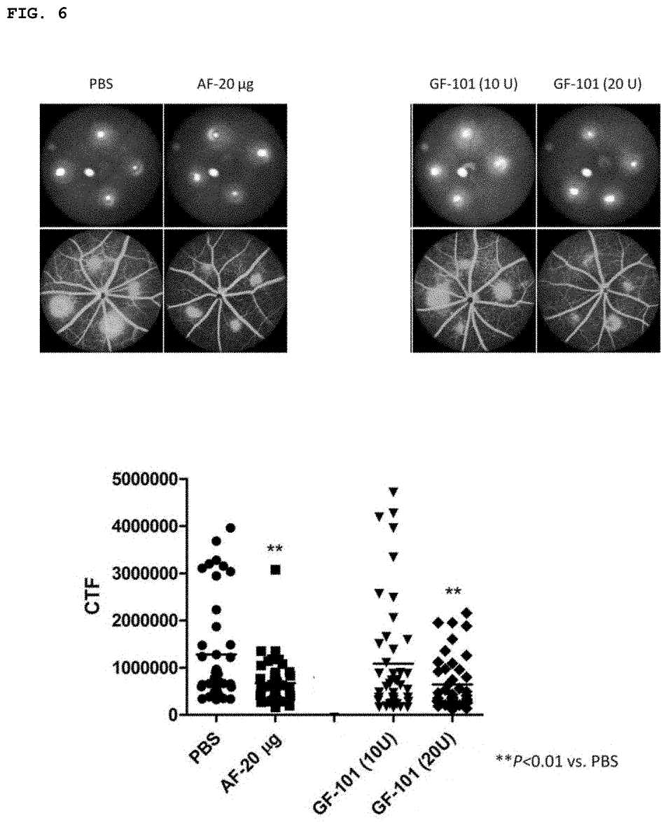

Fluorescein leakage from choroidal neovascularization was measured using fundus fluorescein angiography (FFA). Fundus fluorescent angiography was performed using a micron IV imaging system. 2% fluorescein was injected intraperitoneally into the mice of each test group under anesthesia, and after waiting for 3 to 5 minutes, the pupils were dilated, fundus fluorescein angiography (FFA) imaging was performed, the background was corrected, and the CTF values were calculated. As shown in FIG. 6, it was observed that choroidal neovascularization (CNV) lesions were formed 12 days after laser irradiation.

After administration of the pharmaceutical composition of the present invention, the area of CNV in the eye of the mice, measured by fundus fluorescence angiography, was decreased compared to the CNV area before the start of treatment. The decreased retinal thickness is a decreased central retinal subfield thickness (CST), a decreased center point thickness (CPT), or a decreased central foveal thickness (CFT).

The CTF value of the group administered intraocularly with the positive control aflibercept (AF) (test group III) was 673,595±486,147, compared to that of the PBS-administered group (test group II) (1,279,587±1,094,827), and the CNV area was decreased by 52.6% compared to that of the PBS-administered group. The GF-101 (10 U)-administered group (test group V) (1,124,635±1,249,267) and the GF-101 (20 U)-administered group (test group VI) (645,099±557,005) showed CTF values that were decreased by 12.1% and 49.6%, respectively. Furthermore, it was observed that the CNV lesions in the test group administered with GF-101 (20 U) were significantly decreased compared to the CNV lesions in the PBS-administered group which was the control group (see FIG. 6).

Optical Coherence Tomography (OCT)

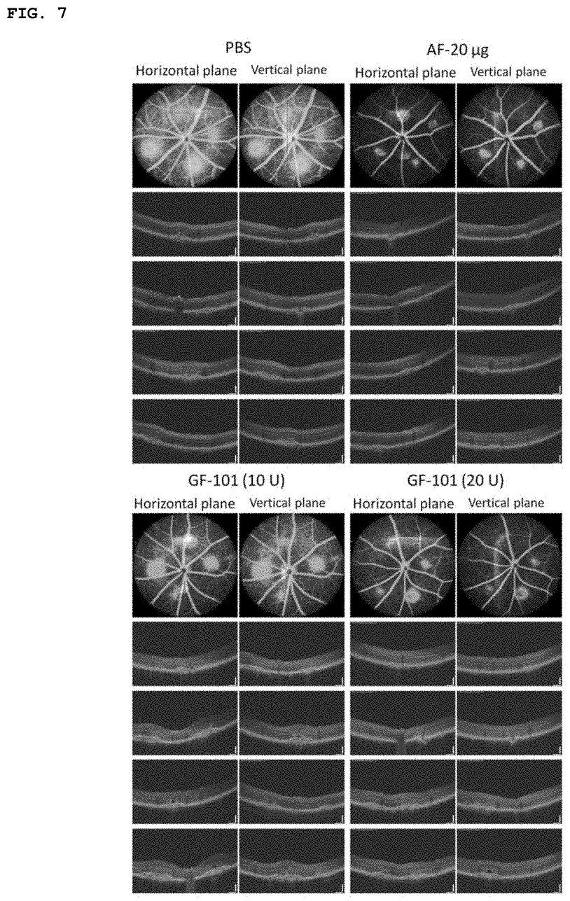

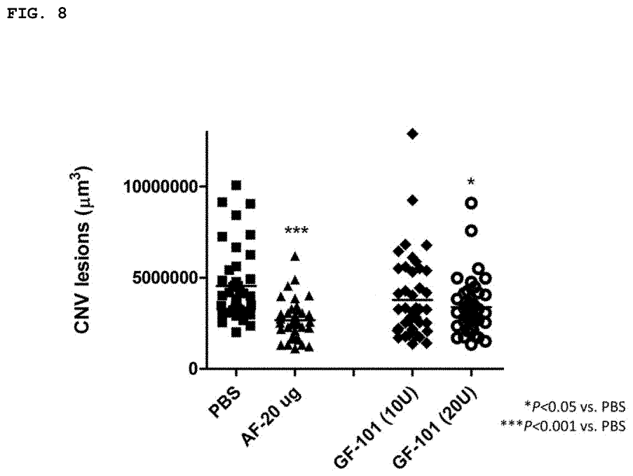

As shown in FIG. 5, on 12 days after laser irradiation, fundus fluorescein angiography imaging was performed, and at the same time, optical coherence tomography (OCT) was performed to obtain the detailed sections and 3D images of the eyes from the mouse retinas. Tomography of each lesion site was performed by transmitting an OCT beam through the center of the CNV lesion on the fundus fluorescein angiography image (FIG. 7), and the image J program was used to quantify the CNV lesion. Retinal tomography was performed by changing the direction of the OCT beam horizontally and vertically for each laser burn. The size of the CNV lesions was measured, and the results are shown in FIG. 8.

The eye retinal thickness of the mice, measured by optical coherence tomography (OCT), was decreased compared to the ocular retinal thickness measured before administration of the compositions of the present invention. Specifically, the size of the CNV lesions was 4,548,182±1,983,055 μm³ in the PBS-administered group (test group II) and was 2,674,277±1,064,973 μm³ in test group III (aflibercept-administered group), which decreased by 41.2% compared to that in the PBS-administered group (test group II). The GF-101 (20 U)-administered group (test group VI) (3,471,454±1,534,395 μm³) showed CNV lesions that decreased by 23.6%, indicating that the CNV lesions were significantly decreased by the administration of GF-101 (20 U).

Electroretinography (ERG)

To evaluate a retinal function, mice were dark-adapted for 24 hours and subjected to electroretinography in the dark on 13 days after laser irradiation. Electroretinography measures the electrical activity produced by photoreceptor cells in the retina when the eye is stimulated by a specific light source. These measurements are recorded through electrodes disposed on the front surface of the eye (e.g., the cornea) and on the skin near the eye, thereby producing a graph called an electroretinogram (ERG).

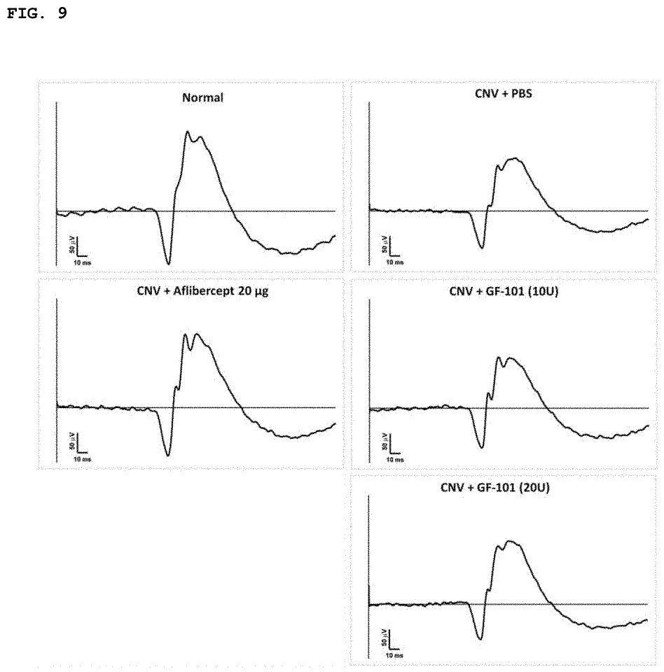

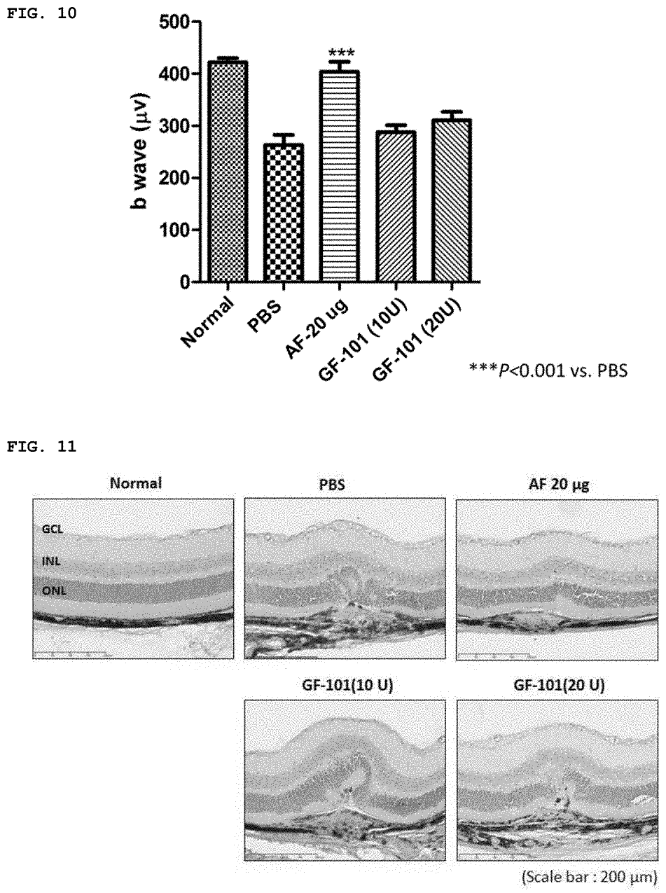

For electroretinography, both eyes of CNV mice were dilated and anesthetized, and then electroretinography was performed by bringing electrodes into contact with the skin, tail, and cornea, respectively. The retina was stimulated by a single white light with a flash intensity of 0.8 cd sec/m² to obtain a response value. The amplitude was measured from the valley of the a-wave to the apex of the b-wave, and the results of the measurement are shown in FIG. 9. The amplitude was evaluated as an indicator of retinal function.

Referring to FIG. 9, the amplitude of the Scotopic b-wave was 263.64±59.88 μV in test group II (PBS-administered group), which decreased by 153.13 μV compared to that of test group I (normal group) (422.27±27.34 μV). The b-wave amplitude of test group III was 403.97±53.79 μV, indicating that the responsiveness of this group was increased by the administration of aflibercept. However, in the case of the GF-101 (10 U)-administered group (test group V) (288.233±37.41 μV), the change in retinal function by drug administration could not be observed. The b-wave amplitude of the group administered with GF-101 (20 U) was 310.80±53.42 μV, indicating that this group had increased responsiveness to light.

Statistical Analysis

The percentage of laser spots with CNV at different doses of a SOD or its 100 kD fragment derived from the B. amyloliquefaciens GF423 strain was compared pair-wise by a chi-square test. The results were plotted against the dose of the SOD derived from the B. amyloliquefaciens GF423 strain to derive the best-fit curve, which was used to calculate the dose of SOD that reduces the fraction of laser spots with CNV by 50% (ED₅₀). A confidence level of p<0.05 was considered statistically significant.

4.4. Histological Analysis

In order to observe the change in tissue by a laser, the mouse eyes were enucleated and fixed with 10% formalin for 10 minutes, and then they were placed in disposable base molds, embedded in an OCT compound, and frozen rapidly in liquid nitrogen.

Hematoxylin & Eosin (H & E) Staining

The tissue samples treated by the above-described method were sectioned, attached to slides, and then dried for about 1 hour, followed by the construction of CNV models. Then, in order to observe the changes in mouse retinas by drug treatment, the samples were stained with hematoxylin & eosin (H & E) and washed. The samples were treated with HCl solution and stained with eosin solution for 30 seconds to 1 minute, and then washed again. The samples were treated with 80%, 85%, 90%, and 100% ethanol for 3 minutes for each treatment, and then reacted with carboxylene and xylene for 5 minutes for each reaction. Next, the embedded tissues were imaged with a virtual microscope (NanoZoomer 2.0 RS), and the images are shown in FIG. 11.

FIG. 11 shows choroidal neovascularization in the eyes (after H & E staining) of the laser-irradiated CNV mice compared to the normal group. In the group administered with PBS after CNV induction, CNV generation was observed together with tissue collapse of the laser-irradiated site. In the GF-101 (10 U)-administered group (test group V), the CNV lesions did not decrease significantly. However, the CNV lesions are decreased in the GF-101 (20 U)-administered group (test group VI).

TUNEL Assay

A TUNEL assay was performed to observe dead cells in the mouse retina after drug treatment in CNV models. Staining was performed using a fluorescence detection TUNEL assay kit. The tissue sections were de-paraffinized with xylene, and then hydrated twice with 100% ethanol, once with 95% ethanol, and once with 85% ethanol in order, followed by washing once with PBS. The tissue surface was wiped clean, and the slides were incubated directly with proteinase K (20 μg/mL) at room temperature for 15 minutes, and then washed twice with PBS. The tissue surface was wiped clean and the slides were incubated directly with 75 μL of equilibration buffer at room temperature for 10 seconds. The tissue surface was wiped clean and the slides were incubated directly with 55 μL of working strength TdT enzyme 37° C. for 1 hour. The slides were washed by shaking with a working strength stop/wash buffer for 15 seconds and then incubated for 10 minutes at room temperature, followed by washing three times with PBS. The tissue surface was wiped clean, incubated directly with 65 μL of an anti-digoxigenin conjugate, and allowed to be left at room temperature for 30 minutes under light-shielded conditions. The slides were washed four times with PBS, stained with DAPI, and then observed with a fluorescence microscope (LEICA DM 2500).

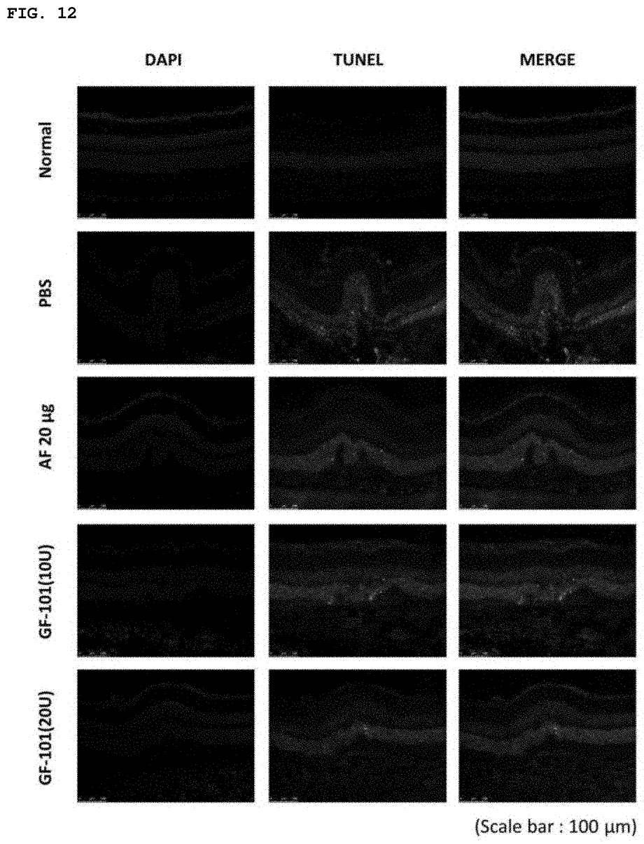

FIG. 12 shows a TUNEL assay performed to observe dead cells in the mouse retina after drug treatment in CNV models. TUNEL response indicative of cell death was observed intensively in the CNV site and in the outer nuclear layer (ONL). The highest number of dead cells was found in the group treated with PBS after CNV induction (test group II), and the number of dead cells in the GF-101 (20 U)-administered group (test group VI) decreased to a level similar to that in the positive control aflibercept-administered group (test group III) (see FIG. 8).

4.5. Results

Blood vessels were stained with fluorescein and subjected to fundus fluorescein angiography. As a result, the GF-101 (20 U)-administered group (test group VI) showed significantly low CTF values.

The results of OCT showed a tendency to the results of fundus fluorescein angiography.

Electroretinography also showed the same tendency. In order to analyze the responsiveness of the retina to light, the mouse eyes were stimulated with different intensity lights at 0.8 log cds/m², and the degree of response to the light was analyzed. It could be seen that the amplitude in the CNV-induced group (test group II) decreased by about 150 μV compared to that in the normal group (test group I), indicating that the retinal function of test group II was declined. The b-wave amplitude of the group administered with GF-101 (20 U) increased in responsiveness to light.

For histological analysis, CNV lesions were analyzed by H & E staining, and photoreceptor cell death in the CNV site was analyzed using TUNEL staining. Increasing CNV size affected the surrounding tissues, and cells damaged in this process were observed in the outer nuclear layer (ONL). However, fewer dead cells were observed in the GF-101 (20 U)-administered group (test group VI).

In summary, it is demonstrated herein that GF-101 (20 U) improved retinal function by effectively suppressing the choroidal neovascularization induced by laser irradiation. In addition, GF-101 (20 U) inhibited cell death, as demonstrated by histopathology and TUNEL assays.

As described above, the compositions of the present disclosure, comprising SOD derived from B. amyloliquefaciens GF423 strain, have excellent antioxidant activity, highly stable enzyme activity, and excellent in vivo stability, and thus can be advantageously used as a material for a pharmaceutical drug, a food, a medical food, etc. for preventing or treating macular degeneration, particularly age-related macular degeneration.

### Example 5. Evaluation of Anti-Inflammatory Effect of Superoxide Dismutase (SOD) Derived from Bacillus amyloliquefaciens GF423

Immuno-Fluorescence (IF) Staining for Inflammation Markers

Inflammation is an important mechanism that promotes CNV formation and immune responses that are associated with its pathogenesis. Therefore, after manufacturing a CNV model, IF staining was performed to detect the presence of inflammatory factors. Animal, CNV induction, dosing regimen, and tissue manipulating methods were performed as described in example 4, except the following doses of GF-103 were used: 20, 40, 60, and 80 SOD units/mouse. Sections were permeabilized with a 0.5% Triton X-100 solution and washed 3 times with PBS for 5 minutes. The Sections were blocked for 1 hour with a blocking solution containing 5% normal serum of the secondary antibody species (goat or donkey), 3% BSA, and 0.5% Triton X-100. Then, the sections were incubated with primary antibodies (see Table 3) in PBS including 3% BSA and 0.5% Triton X-100 at 4° C. overnight.

TABLE 3

| Primary | | | | Secondary |
|---|---|---|---|---|
| antibody | Host | Dilution | Specimen | antibody |
| ICAM-1 | Rabbit | 1:500 | Cryo- | Alexa Fluor 555 Donkey |
| | | | section | anti-Rabbit IgG |
| CD45 | Rat | | | Alexa Fluor 647 goat |
| F4/80 | Rat | | | anti-Rat IgG |

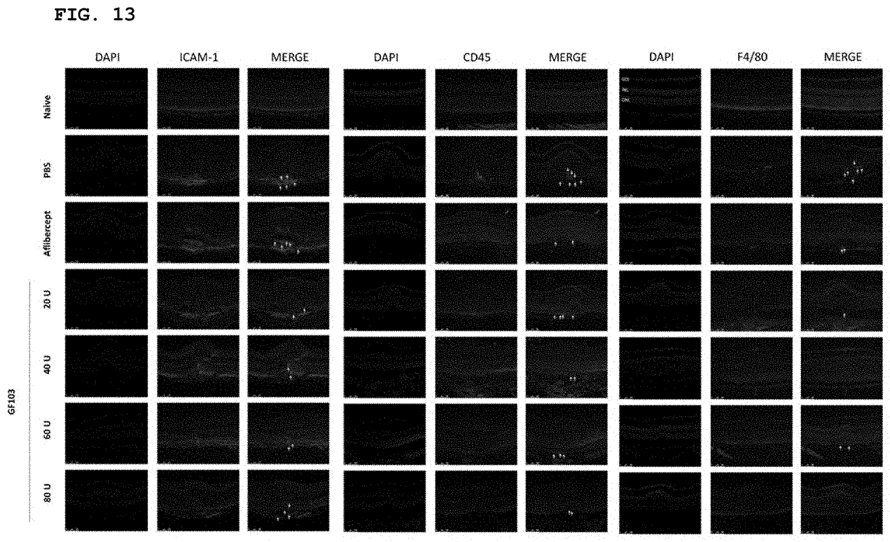

While ICAM-1 and CD45 (general leukocyte markers), and F4/80 (a marker of murine macrophage) were observed at the outer nuclear layer and CNV lesion in the PBS group (FIG. 13), they were significantly decreased in the aflibercept and GF-103 treatment groups (FIG. 13). The results indicate that GF-103 administration decreases infiltration of immune cells such as leukocytes and macrophages in the CNV region, leading to anti-inflammatory effects, shown as a decrease in the ICAM-1 level.

### Example 6. Dose-Response Relationship of SOD Treatment to Choroidal Neovascularization (CNV) Lesion, Retinal VEGF, and Hif-1-Alpha in the Retina

6.1. Experimental Animals and Construction of Choroidal Neovascular (CNV) Models

Animal experiments were performed in accordance with the Animal Use and Care Protocol of the Institutional Animal Care and Use Committee (IACUC). Brown Norway (BN) rats were purchased from Central Lab Animal Inc. (5F Gun B/D, Baumoe-ro 7-gil, Seocho-gu, Seoul, 137-900 Korea), and acclimated for 5 days. Then, the rats were raised for 14 days at an average temperature of 21.0 to 24.2° C., the humidity of 53.5 to 67.1%, and an average illuminance of 150 to 300 lux with a 12-hr light/12-hr dark cycle. The rats were given feed and water ad libitum daily.

8-week-old BN rats were anesthetized by the intraperitoneal injection of Alfaxan (Jurox, Australia, 3 mL/kg) and Rompun (Bayer Korea, 0.5 mL/kg). Under anesthesia, pupils were dilated using mydriatics, and three or four photocoagulation spots were created around the optic nerve head via a diode laser (Oculight Glx, Iridex Inc, CA, USA). The equipment set-up condition was as follows.

wavelength: 532 nm

diameter: 100 μm

power: 220 mW

duration: 0.1 sec.

The destruction of Bruch's membrane was confirmed by characteristic bubble formation.

6.2. Administration of Test Substances

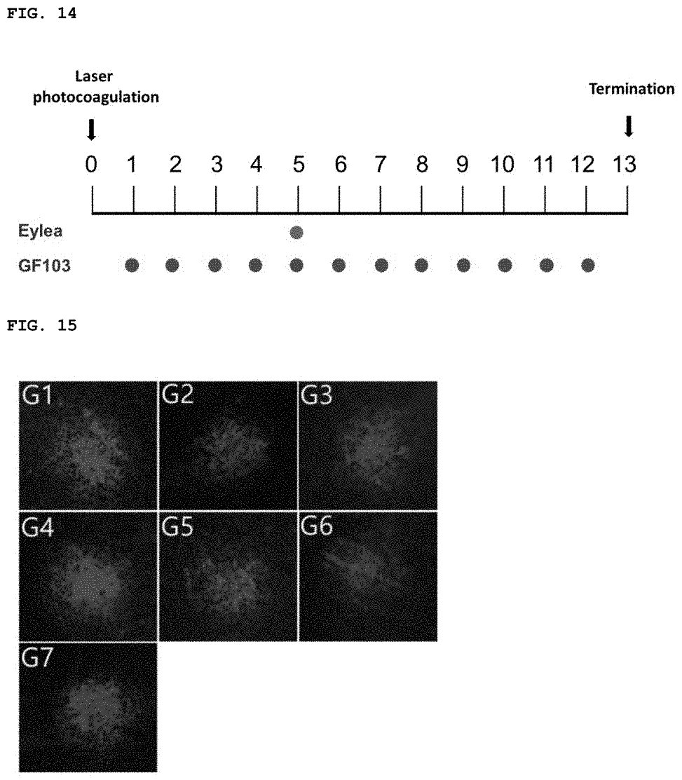

Experimental animals were grouped as described in Table 4. One day after laser irradiation, GF-103 suspended in PBS was administered orally daily for 14 days. Aflibercept was administered once on day 5 by Intraocular (vitreous body) injection. To a negative control group, PBS was administered as a placebo in the same way as the GF-101 treatment group (FIG. 14).

TABLE 4

| | | | | | The number |
|---|---|---|---|---|---|
| | Experimental group | Dose | | Route | of animals |
| G1 | CNV (negative | — | | PO | 7 |
| | control group) | | | | |
| G2 | Aflibercept (positive | 20 | μg/eye | IVT | 7 |
| | control group) | | | | |
| G3 | GF-103 test group | 50 | SOD Units/kg | PO | 7 |
| G4 | GF-103 test group | 100 | SOD Units/kg | PO | 7 |
| G5 | GF-103 test group | 250 | SOD Units/kg | PO | 7 |
| G6 | GF-103 test group | 500 | SOD Units/kg | PO | 7 |
| G7 | GF-103 test group | 1,000 | SOD Units/kg | PO | 7 |

6.3. Animal Test

CNV Formation Evaluation

After fluorescein retinal angiography, an autopsy was carried out on animals to evaluate the CNV formation. At autopsy, the eye was extracted, and the eyeball was incised at the area adjacent to the cornea and sclera under the microscope. The retina was detached from the back portion of the eyeball, and then conjunctival tissue including the subretina was separated.

Immunofluorescence staining was performed for the extracted tissue using isolectin B4 (Sigma-Aldrich, USA), which is an endothelial cell marker. Stained tissue was examined under the fluorescent microscope (BX51, Olympus, Japan), and the size of CNV was analyzed via Image J software (NIH, USA).

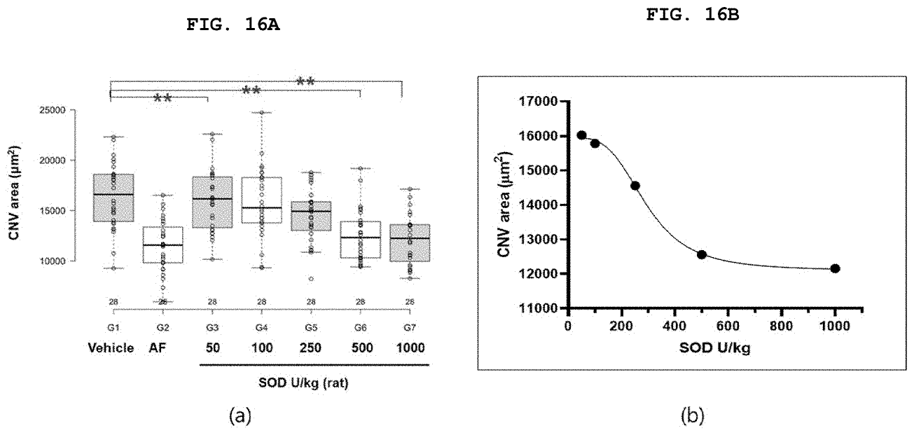

GF-103 administration resulted in a statistically significant decrease in CNV lesions (FIGS. 15 and 16A). Specifically, the area of the lesion in the negative control group (G1) was 16,488±3,262 μm²; the positive control group (G2) was 11,485±2,572 μm²; the GF-103 test group (G6) was 12,560±2,547 μm²; and the GF-103 test group (G7) 12,158±2,440 μm². In addition, the CNV areas in the GF-103 treatment groups decreased in a dose-dependent manner (FIG. 16B).

ELISA Analysis (Hif-1-Alpha and VEGF)

The retina was extracted from the other side of the eyeball not used for CNV formation, and the presence of VEGF (vascular endothelial growth factor) and HIF-1-alpha (Hypoxia-inducible factors-1-alpha) was examined by ELISA. After eyeball extraction at autopsy, under a dissecting microscope, the retina was carefully separated from the eyeball, and then washed with PBS and stored at −70° C. until analysis. To extract proteins for the ELISA analysis, the retina was weighed. After adding 5 times the weight of lysis buffer (MyBiosource, San Diego, Calif., USA), it was homogenized by a bead homogenizer (BioPrep-24, Allsheng, Hangzhou City, cycles of 6 m/sec, 20 sec each). The homogenized tissue was spun down at 1000×g for 5 minutes at 4° C., and the supernatant was used for the protein analysis. The total protein amount for the sample was quantified by the Bradford (Bio-rad, USA) method. ELISA kit information is as follows.

VEGF: Rat VEGF ELISA kit, Abcam, Lot No.: GR3355645-1.

HIF-1: Rat hypoxia-inducible factor 1alpha ELISA kit, MyBioSource, Lot No.: W24141530.

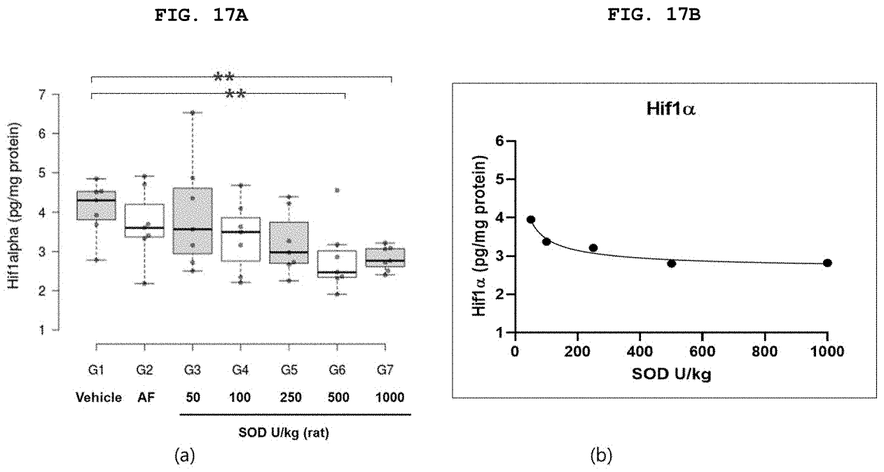

GF-103 administration at a dose of 500 and 1000 unit/kg significantly reduced the level of Hif-1-alpha in retina tissue, compared to the PBS group (FIG. 17a). The level of Hif-1-alpha in the GF-103 treatment groups decreased in a dose-dependent manner (FIG. 17b)

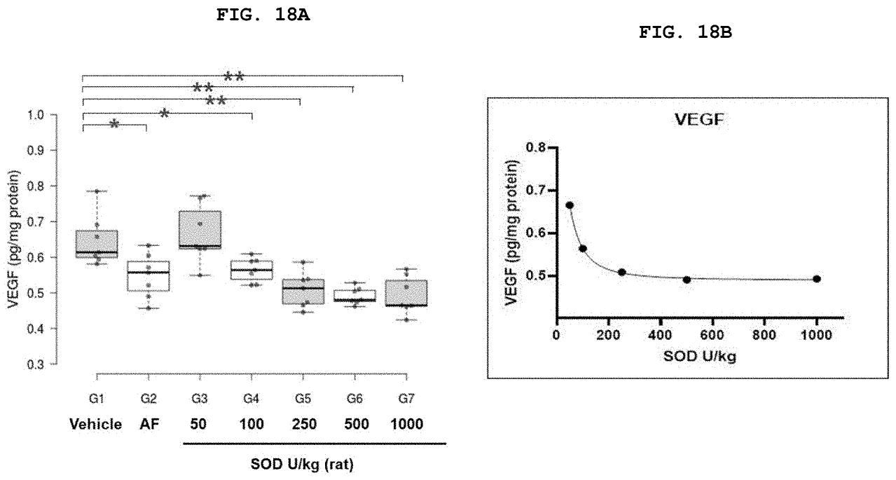

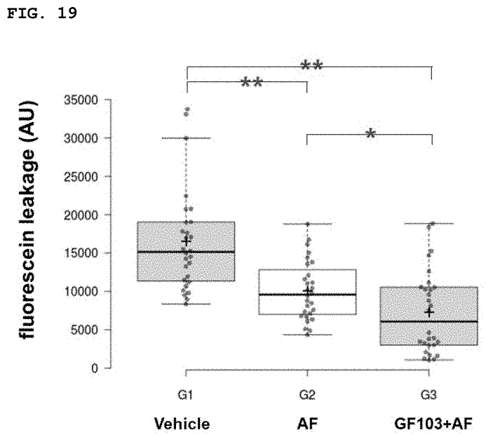

GF-103 administration at a dose of 100 unit/kg or higher significantly reduced the level of VEGF in retina tissue, compared to the PBS group (FIG. 18A). Aflibercept administration also reduced the level of VEGF. The level of VEGF in the GF-103 treatment groups decreased in a dose-dependent manner (FIG. 18B).

### Example 7. Effect of the Combination of GF-103 and Aflibercept on Inhibition of Choroidal Neovascularization

7.1. Experimental Animals and Construction of Choroidal Neovascular (CNV) Models

Animal experiments were performed as described in Example 6.

7.2. Administration of Test Substances

Experimental animals were grouped as described below (Table 5). Aflibercept was administered once on day 5 by intraocular (vitreous body) injection (G2). For the combination group (G3), GF-103 suspended in PBS was administered orally daily from day −6 to day 12 for 19 days; and Aflibercept was administrated the same way as the G2 group. To a CNV-induced group (G1), PBS was administered as a placebo in the same way as GF-103.

TABLE 5

| | Experimental | | | | The number |
|---|---|---|---|---|---|
| | group | Dose | | Route | of animals |
| G1 | CNV (negative | — | | PO | 7 |
| | control group) | | | | |
| G2 | Aflibercept | 20 | μg/eye | IVT | 7 |
| G3 | GF-103 + | 50 | SOD Units/kg | PO, IVT | 7 |
| | Aflibercept | | | | |

7.3. Animal Test

Fluorescein Retinal Angiography

On the day of autopsy, the animals were intraperitoneally injected with Alfaxan (Jurox, Australia, 3 mL/kg) and Rompun (Bayer Korea, 0.5 mL/kg) to induce anesthesia, and then the pupils were dilated with 1% tropicamide eye drop. During anesthesia and pupil dilation, the amount of anesthetic and mydriasis was increased or decreased depending on the depth of anesthesia and pupil dilation. Under anesthesia, approximately 1 mL blood was collected from the caudal vena cava or jugular vein. Sodium fluorescein (Sigma, MO, USA) was dissolved in vehicle substance (PBS) at a concentration of 10%, and then 1 ml/kg was injected to the abdominal vena cava. After about 5 minutes, an image of the fundus oculi was taken with a fundus microscope (Fluorescence endoscope, Karl Storz, Tuttlingen, Germany). Fluorescein leakage intensity was analyzed using Image J software (NIH, USA).

As a result of fluorescein retinal angiography, the fluorescein leakage intensity of the aflibercept and combination group was significantly reduced. The leakage in the combination group was reduced more than the leakage in the aflibercept group, indicating that the combination therapy is more effective than monotherapy of aflibercept (FIG. 19).

The description provided herein is illustrative of preferred embodiments and is not intended to limit the scope of the present invention. It will be obvious to those skilled in the art that various modifications and changes are possible without departing from the spirit and scope of the present invention.

### Accession Numbers

Depository authority: the Korea Research Institute of Bioscience and Biotechnology

KCTC13222BP

Deposit date: Mar. 6, 2017

What is claimed is:

1. A method of treating or preventing macular degeneration, comprising administering to a subject in need thereof a composition comprising a superoxide dismutase (SOD) enzyme; or
a method of decreasing or inhibiting choroidal neovascularization (CNV) by contacting a retina with a composition comprising a SOD enzyme.

2.-3. (canceled)

4. The method of claim 1, wherein the SOD enzyme comprises:
(a) the amino acid sequence with at least or about 85% identity to the sequence set forth in SEQ ID NO: 1 or SEQ ID NO: 38;
(b) the amino acid sequence set forth in SEQ ID NO: 1 or SEQ ID NO: 38, wherein the amino acid residue Asn74 and/or Asn137 is deleted or substituted;
(c) the amino acid sequence set forth in SEQ ID NO: 1 or SEQ ID NO: 38, wherein the amino acid residue Asn74 and/or Asn137 is substituted with Asp74 and/or Asp137; or
(d) the amino acid sequence set forth in SEQ ID NO: 1 or SEQ ID NO: 38.

5. The method of claim 1, wherein the SOD enzyme (i) is an isolated enzyme, (ii) binds manganese, and/or (iii) is coated with shellac.

6. The method of claim 1, wherein the composition is administered orally, intravenously, intraocularly, or intramuscularly, optionally wherein the composition is administered orally.

7. (canceled)

8. The method of claim 1, wherein the SOD enzyme is from a microorganism, preferably a bacterium, preferably a bacterium generally regarded as safe (GRAS) for use as food, more preferably a Bacillus species bacterium.

9. The method of claim 8, wherein the SOD enzyme is from Bacillus amyloliquefaciens GF423 strain (KCTC 13222BP).

10. The method of claim 1, wherein the composition
(i) decreases choroidal neovascularization (CNV);
(ii) decreases cell death in the retina;
(iii) decreases inflammation in the retina;
(iv) decreases the expression of vascular endothelial growth factor (VEGF) in the retina;
(v) decreased the expression of Hypoxia-inducible factor 1-alpha (HIF-1-alpha) in the retina; and/or
(vi) increases the retinal function.

11. The method of claim 1, wherein the macular degeneration is an age-related macular degeneration (AMD), preferably wherein the AMD is a wet AMD or a neovascular AMD.

12. The method of claim 1, wherein the composition is a pharmaceutical composition or a nutraceutical food.

13. The method of claim 1, further comprising administering at least one additional agent that treats macular degeneration; or decreases or inhibits CNV, optionally wherein the at least one additional agent is ranibizumab or aflibercept.

14. (canceled)

15. The method of claim 1, wherein the subject is a mammal, preferably wherein the mammal is a human, a dog, a cat, a mouse, or a rat, optionally wherein the subject is a human.

16.-26. (canceled)

27. An engineered polypeptide, a pharmaceutical composition, or a medical or nutraceutical food comprising a superoxide dismutase (SOD) enzyme.

28-30. (canceled)

31. The engineered polypeptide, the pharmaceutical composition, or the medical or nutraceutical food of claim 27, wherein the SOD enzyme comprises:
(a) the amino acid sequence with at least or about 85% identity to the sequence set forth in SEQ ID NO: 1 or SEQ ID NO: 38;
(b) the amino acid sequence set forth in SEQ ID NO: 1 or SEQ ID NO: 38, wherein the amino acid residue Asn74 and/or Asn137 is deleted or substituted;
(c) the amino acid sequence set forth in SEQ ID NO: 1 or SEQ ID NO: 38, wherein the amino acid residue Asn74 and/or Asn137 is substituted with Asp74 and/or Asp137; or
(d) the amino acid sequence set forth in SEQ ID NO: 1 or SEQ ID NO: 38.

32. The engineered polypeptide, the pharmaceutical composition, or the medical or nutraceutical food of claim 27, wherein the SOD enzyme (i) is an isolated enzyme, (ii) binds manganese, and/or (iii) is coated with shellac.

33. The engineered polypeptide, the pharmaceutical composition, or the medical or nutraceutical food of claim 27, wherein the pharmaceutical composition is an oral composition.

34. The engineered polypeptide, the pharmaceutical composition, or the medical or nutraceutical food of claim 27, wherein the SOD enzyme is from a microorganism, preferably a bacterium, preferably a bacterium generally regarded as safe (GRAS) for use as food, more preferably a Bacillus species bacterium.

35. The engineered polypeptide, the pharmaceutical composition, or the medical or nutraceutical food of claim 34, wherein the SOD enzyme is from Bacillus amyloliquefaciens GF423 strain (KCTC 13222BP).

36. The engineered polypeptide, the pharmaceutical composition, or the medical or nutraceutical food of claim 27, wherein the engineered polypeptide, pharmaceutical composition, or medical or nutraceutical food
(i) decreases choroidal neovascularization (CNV);
(ii) decreases cell death in the retina;
(iii) decreases inflammation in the retina;
(iv) decreases the expression of vascular endothelial growth factor (VEGF) in the retina;
(v) decreased the expression of Hypoxia-inducible factor 1-alpha (HIF-1-alpha) in the retina; and/or
(v) increases the retinal function.

37. The engineered polypeptide, the pharmaceutical composition, or the medical or nutraceutical food of claim 27, further comprising at least one additional agent that decreases or inhibits CNV in the retina, optionally wherein the at least one additional agent is ranibizumab or aflibercept.

38.-49. (canceled)

50. A kit comprising the engineered polypeptide, the pharmaceutical composition, or the medical or nutraceutical food of claim 27.


SEQUENCE LISTING (38 sequences)

SEQ ID NO: 1 - protein, 201 aa, Bacillus amyloliquefaciens
MAYKLPELPYAYDALEPHIDKETMTIHHTKHHNTYVTNLNKAIEGSALAEKSVDELVADLNAVPEDIRTAVRNNGGGHANHSLFWTLLSPNGGGEPTGELAEEIKSTFGSFDQFKEKFAAAAAGRFGSGWAWLVVNNGKLEITSTPNQDSPLSEGKTPVLGLDVWEHAYYLNYQNRRPDYISAFWNVVNWDEVARLYSEAK

SEQ ID NO: 2 - DNA, 1543 bp, Bacillus amyloliquefaciens
agagtttgatcctggctcaggacgaacgctggcggcgtgcctaatacatgcaagtcgagcggacagatgggagcttgctccctgatgtcagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 3 - DNA, 1543 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaatagcggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 4 - DNA, 1543 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 5 - DNA, 1543 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 6 - DNA, 1543 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggatgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 7 - DNA, 1543 bp, Bacillus amyloliquefaciens; identical to SEQ ID NO: 6

SEQ ID NO: 8 - DNA, 1543 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 9 - DNA, 1543 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaatcgcatggttcagacataaaaggtggcttctgctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 10 - DNA, 1543 bp, Bacillus amyloliquefaciens
agagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccagatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 11 - protein, 17 aa, Artificial Sequence
  Description of Artificial Sequence Synthetic peptide
LPELPYAYDALEPHIDK

SEQ ID NO: 12 - protein, 9 aa, Artificial Sequence
  Description of Artificial Sequence Synthetic peptide
ETMTIHHTK

SEQ ID NO: 13 - protein, 26 aa, Artificial Sequence
  Description of Artificial Sequence Synthetic peptide
LPELPYAYDALEPHIDKETMTIHHTK

SEQ ID NO: 14 - protein, 20 aa, Artificial Sequence
  Description of Artificial Sequence Synthetic peptide
LPELPYAYDALEPHIDKETM

SEQ ID NO: 15 - protein, 29 aa, Artificial Sequence
  Description of Artificial Sequence Synthetic peptide
AYKLPELPYAYDALEPHIDKETMTIHHTK

SEQ ID NO: 16 - protein, 11 aa, Artificial Sequence
  Description of Artificial Sequence Synthetic peptide
HHNTYVTNLNK

SEQ ID NO: 17 - protein, 10 aa, Artificial Sequence
  Description of Artificial Sequence Synthetic peptide
AIEGSALAEK

SEQ ID NO: 18 - protein, 17 aa, Artificial Sequence
  Description of Artificial Sequence Synthetic peptide
SVDELVADLNAVPEDIR

SEQ ID NO: 19 - protein, 4 aa, Artificial Sequence
  Description of Artificial Sequence Synthetic peptide
TAVR

SEQ ID NO: 20 - protein, 33 aa, Artificial Sequence
  Description of Artificial Sequence Synthetic polypeptide
NDGGGHANHSLFWTLLSPNGGGEPTGELAEEIK

SEQ ID NO: 21 - protein, 10 aa, Artificial Sequence
  Description of Artificial Sequence Synthetic peptide
STFGSFDQFK

SEQ ID NO: 22 - protein, 12 aa, Artificial Sequence
  Description of Artificial Sequence Synthetic peptide
STFGSFDQFKEK

SEQ ID NO: 23 - protein, 8 aa, Artificial Sequence
  Description of Artificial Sequence Synthetic peptide
FAAAAAGR

SEQ ID NO: 24 - protein, 14 aa, Artificial Sequence
  Description of Artificial Sequence Synthetic peptide
FGSGWAWLVVNDGK

SEQ ID NO: 25 - protein, 17 aa, Artificial Sequence
  Description of Artificial Sequence Synthetic peptide
LEITSTPNQDSPLSEGK

SEQ ID NO: 26 - protein, 31 aa, Artificial Sequence
  Description of Artificial Sequence Synthetic polypeptide
FGSGWAWLVVNDGKLEITSTPNQDSPLSEGK

SEQ ID NO: 27 - protein, 20 aa, Artificial Sequence
  Description of Artificial Sequence Synthetic peptide
TPVLGLDVWEHAYYLNYQNR

SEQ ID NO: 28 - protein, 19 aa, Artificial Sequence
  Description of Artificial Sequence Synthetic peptide
RPDYISAFWNVVNWDEVAR

SEQ ID NO: 29 - protein, 6 aa, Artificial Sequence
  Description of Artificial Sequence Synthetic peptide
LYSEAK

SEQ ID NO: 30 - protein, 33 aa, Artificial Sequence
  Description of Artificial Sequence Synthetic polypeptide
  MOD_RES (2)..(2): Deamidated Asn
NNGGGHANHSLFWTLLSPNGGGEPTGELAEEIK

SEQ ID NO: 31 - protein, 33 aa, Artificial Sequence; identical to SEQ ID NO: 30
  Description of Artificial Sequence Synthetic polypeptide
  MOD_RES (1)..(1): Deamidated Asn

SEQ ID NO: 32 - protein, 17 aa, Artificial Sequence
  Description of Artificial Sequence Synthetic peptide
  MOD_RES (1)..(1): Deamidated Asn
NNGGGHANHSLFWTLLS

SEQ ID NO: 33 - protein, 14 aa, Artificial Sequence
  Description of Artificial Sequence Synthetic peptide
PNGEPTGELAEEIK

SEQ ID NO: 34 - protein, 14 aa, Artificial Sequence
  Description of Artificial Sequence Synthetic peptide
  MOD_RES (12)..(12): Deamidated Asn
FGSGWAWLVVNNGK

SEQ ID NO: 35 - protein, 14 aa, Artificial Sequence
  Description of Artificial Sequence Synthetic peptide
  MOD_RES (11)..(11): Deamidated Asn
FGSGWAWLVVNNGK

SEQ ID NO: 36 - protein, 11 aa, Artificial Sequence
  Description of Artificial Sequence Synthetic peptide
FGSGWAWLVVN

SEQ ID NO: 37 - protein, 12 aa, Artificial Sequence
  Description of Artificial Sequence Synthetic peptide
  MOD_RES (10)..(10): Deamidated Asn
SGWAWLVVNNGK

SEQ ID NO: 38 - protein, 200 aa, Artificial Sequence
  Description of Artificial Sequence Synthetic polypeptide
  MOD_RES (72)..(73): Deamidated Asn
  MOD_RES (135)..(136): Deamidated Asn
AYKLPELPYAYDALEPHIDKETMTIHHTKHHNTYVTNLNKAIEGSALAEKSVDELVADLNAVPEDIRTAVRNNGGGHANHSLFWTLLSPNGGGEPTGELAEEIKSTFGSFDQFKEKFAAAAAGRFGSGWAWLVVNNGKLEITSTPNQDSPLSEGKTPVLGLDVWEHAYYLNYQNRRPDYISAFWNVVNWDEVARLYSEAK